(19) United States
(12) Patent Application Publication
(10) Pub. No.: US 2006/0135876 A1
(43) Pub. Date: Jun. 22, 2006

(54) Combined ECG and sound chart report and methodology

(76) Inventors: Alan V. Andresen, McMinnville, OR (US); Peter M. Galen, Portland, OR (US); Robert A. Warner, Tigard, OR (US); Ronald H. S. Selvester, Long Beach, CA (US)

Correspondence Address: ROBERT D. VARITZ, P.C., 4915 S.E. 33RD PLACE, PORTLAND, OR, 97202, US

(21) Appl. No.: 11/140,010

(22) Filed: May 26, 2005

Related U.S. Application Data
(63) Continuation-in-part of application No. 10/607,845, filed on Jun. 27, 2003.

(51) Int. Cl. A61B 5/04

(52) U.S. Cl. 600/513; 600/514; 600/528

(57) ABSTRACT

A computer-based method for presenting correlated ECG and heart-associated sound information regarding a selected subject's heart including the steps of (a) collecting subject-specific, time-related ECG and heart-associated sound information, and thereafter (b), presenting, in a common-display manner, at least portions of this ECG and sound information along with (a) a stated heart-condition interpretation derived from the information, coupled with (b) the presentation of a related, authoritative clinical-interpretation guideline.

### CROSS REFERENCE TO RELATED APPLICATION

This application is a continuation in part from prior-filed, currently co-pending U.S. patent application Ser. No. 10/607,845, filed Jun. 27, 2003 for “Method and System for Detection of Heart Sounds”. The present application claims priority to the filing date of this earlier-filed application, and the entire disclosure contents of that prior application are hereby incorporated herein by reference.

### BACKGROUND AND SUMMMARY OF THE INVENTION

This invention pertains to the gathering and presenting of a subject's ECG and associated heart-related sound information. More particularly, it pertains to the presenting, in a commonly viewable manner, as on a printed chart, on a display screen, or in a stored and later accessible display-relevant database, of what is referred to herein as an integrated report which commonly shows a generated interpretation in text form (based on the gathered information), an ECG/sound-based graphical snippet, and a textual guide to the use of the interpretation information in the form of reference text drawn directly and electronically from an authoritative and recognized, published clinical interpretive guideline set which has been previously developed regarding the general kind and content of such gathered ECG and heart-sound data.

The presentation which is created and made available for viewing in accordance with the present invention neatly and succinctly makes available to the “viewer” a comprehensive, integrated and easily grasped and pictured assessment of a subject's specific heart condition which has produced the ECG/sound snippet and the textually presented interpretation (the findings). Two previously issued U.S. Patents provide useful and interesting background information which is generally associated with the present invention. The contents of these several materials are also hereby incorporated herein by reference. These patents include U.S. Pat. Nos. 6,30,048 B1, and 6,516,220 B2.

As will be seen from a reading of the description of the invention set forth below along with the several included drawings figures, the present invention, in its preferred and best-mode forms, involves either all, or some, of the steps of (a) gathering, in a computer-based setting, time-base-related, associated, selected-subject ECG and sound information, (b) processing such collected information and generating from such processing a reportable interpretation, (c) comparing the generated interpretation with a recognized and authoritative ECG/heart-sound guideline database for the purpose of identifying guideline text which is relevant to the generated interpretation, and (d), as an ultimate consequence of these several predecessor activities, producing an integrated report which is readily viewable by an observer, and which commonly shows the generated interpretation along with the identified, authoritative guideline text.

A preferred practice regarding presentation of information based upon implementation of the invention involves the generation of a strip-chart-like report containing (a) graphical ECG and audio information, (b) interpretative text (an interpretation) based upon computer-implemented processing and analysis of acquired data, (c) a graphical snippet illustrating ECG and audio information derived essentially from a single beat of a subject's heart, and which information has also been used to generate the interpretative text, and (d) confirmatory text drawn from an authoritative body of interpretative guidelines with which the interpretation that has been produced based upon a subject's data has been compared. The integrated report thus provided, “sitting” at a relatively high confidence level because of the underlying comparison and presentation of confirmatory guidelines text, offers a very useful tool for learning much about the condition of a subject's heart.

These and various other features and advantages of the present invention will become more fully apparent as a preferred and best-mode manner of practicing the invention is now set forth below.

### DESCRIPTION OF THE DRAWINGS

FIG. 1 is a high-level, block/schematic drawing illustrating a preferred and best-mode embodiment of, and manner of practicing, the present invention.

FIG. 2 is also a high-level, block/schematic drawing further illustrating a portion of what is shown in FIG. 1.

FIG. 3 is yet another high-level, block/schematic diagram further showing a part of what appears in FIG. 1.

FIG. 4 presents a chart-display report illustrating performance of the invention.

FIG. 5, appearing on the fifth plate of drawings, illustrates guideline text which is directly associated with the report of FIG. 4.

FIG. 6 presents another chart-display report illustrating performance of the present invention.

FIG. 7, also found on plate five of the drawings, relates to FIG. 6 in the same way that FIG. 5 relates to FIG. 4.

### DETAILED DESCRIPTION OF THE INVENTION

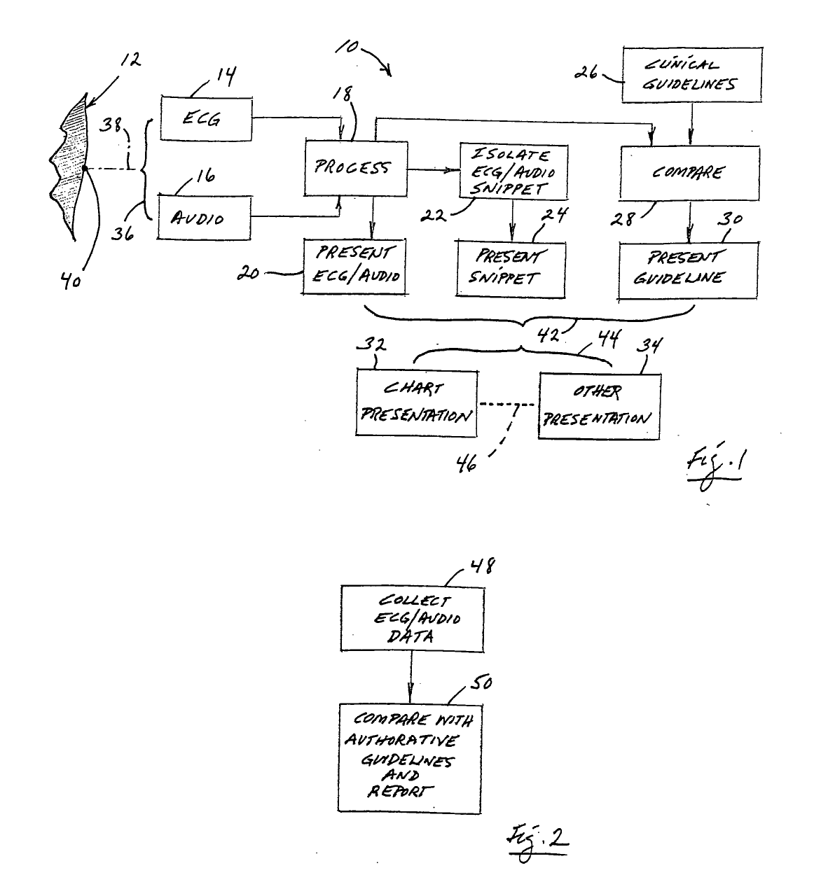

Turning now to the drawings, and referring first of all to FIG. 1, shown generally at 10 in this figure are both a system and a methodology which are computer based, and which are designed to implement the present invention involving the acquisition and presentation of correlated ECG and heart-associated sound information regarding a selected subject's heart. Shown fragmentarily, and somewhat shaded, at the left side of FIG. 1, at 12, is a portion of a human subject's anatomy.

Included in FIG. 1 are a block 14 labeled ECG, a block 16 labeled AUDIO, a block 18 labeled PROCESS, a block 20 labeled PRESENT ECG/AUDIO, a pair of blocks 22, 24, labeled, respectively, ISOLATE ECG/AUDIO SNIPPET and PRESENT SNIPPET, and a trio of blocks 26, 28, 30 labeled, respectively, CLINICAL GUIDELINES, COMPARE, and PRESENT GUIDELINE. Further included in FIG. 1 are two more blocks 32, 34 labeled, respectively, CHART PRESENTATION and OTHER PRESENTATION.

Obvious arrow-headed lines in FIG. 1 illustrate operative processing flow connections between different ones of these just-mentioned blocks. A bracket 36, a dash-dot line 38, and a large blackened dot 40, which appear toward the left side of FIG. 1, represent an operative data-collection coupling between the blocks of methodology 10 and the subject anatomy 12. A pair of brackets 42, 44 represent an operative association between blocks 32, 34, and blocks 14-30, inclusive. A short dashed line 46 extends as shown between blocks 32, 34 for a reason which will be explained shortly.

Blocks 14, 16 represent a suitable plurality of ECG and audio transducers which are appropriately coupled to anatomy 12 so as to collect both a standard 12-lead set of ECG waveforms, and at least one audio signal, collectively reflective of heart activity. According to practice of the invention, ECG and audio signals collected, preferably on a real-time basis, are fed to block 18 wherein appropriate computer processing takes place to perform an interpretation (findings) of the thus-colleted, common-time-base, ECG and audio information, all for the purpose of producing an interpretation relevant to a particular subject's current heart behavior. Computer-based algorithms which are and may be employed to accomplish this do not form any part of the present invention, and thus are not discussed herein in any detail.

The processing which is performed block 18 produces what is referred to herein as a reportable interpretation which is furnished through block 20, and thereafter to one or both of blocks 32, 34, to a visual display of both ECG and sound information, as well as text information describing the interpretation which has been produced by the operation of block 18.

Digressing for just a moment to the region in FIG. 1 occupied by blocks 32, 34, block 32 represents a visual display output in the form of what might otherwise be considered to be a conventional strip-chart document. Block 34 represents any other suitable form enabling visual presentation, such as an appropriate display screen, and a storable, display-relevant database which may be retained and brought forth at later times, if desired, for the purpose of creating a display relevant to a particular collection of a patient's ECG and heart-sound data. In the description of the preferred manner of practicing the invention now being described herein, display output is illustrated, as will shortly be explained, in FIGS. 4-7, inclusive, in the form of a strip-chart display report. Dashed line 46 simply indicates that one or more of various “output-reception” devices/instrumentalities may be used.

Based upon the interpretive processing performed by block 18, blocks 22, 24 are brought into play, with block 22 isolating, and block 24 presenting for display output, what is referred to herein as a combined ECG and audio or sound snippet. This snippet, in accordance with preferred practice of the invention, effectively presents, on a common time base, related ECG and audio information which takes place roughly through the duration of a single subject heartbeat. This snippet is directly associated with the reportable interpretation that has been generated by block 18. One way of thinking about this operation is that the interpretive activity performed in and by block 18 makes a selection of a fragment of an EGC waveform derived from one of the twelve leads of information gathered, along with time-simultaneous audio information, thus to create a graphical representation of the interpretation which, as was just mentioned above, is also output in text form.

A further procedure performed in accordance with one significant manner of practicing the invention takes place via the collaborative operations of blocks 18, 26, 28, 30. Block 26 represents a conventionally available electronic database of recognized, authoritative clinical interpretation textual guidelines, such as the guidelines made available in ACC/AHA Guidelines for Management Of Patients with Acute MI. Information developed as an interpretation is compared, or related, in block 28 with the content in database 26 for the purpose of locating a recognized specific guideline which is associated with the computer-based interpretation that has been produced. Such a guideline, or guidelines if plural guidelines are found to be relevant, is/are presented through block 30 to the display output of the system—in the case now being described, to a printed strip-chart-type report.

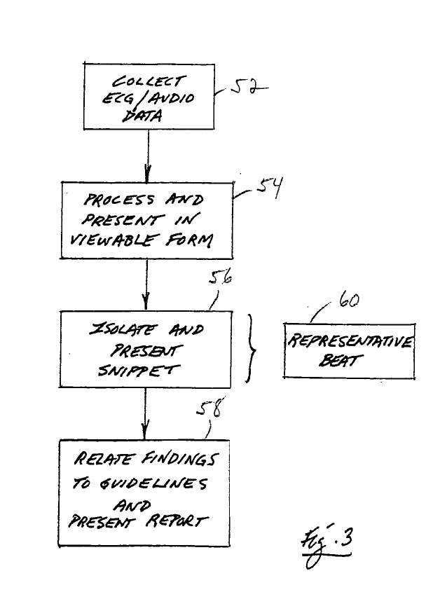

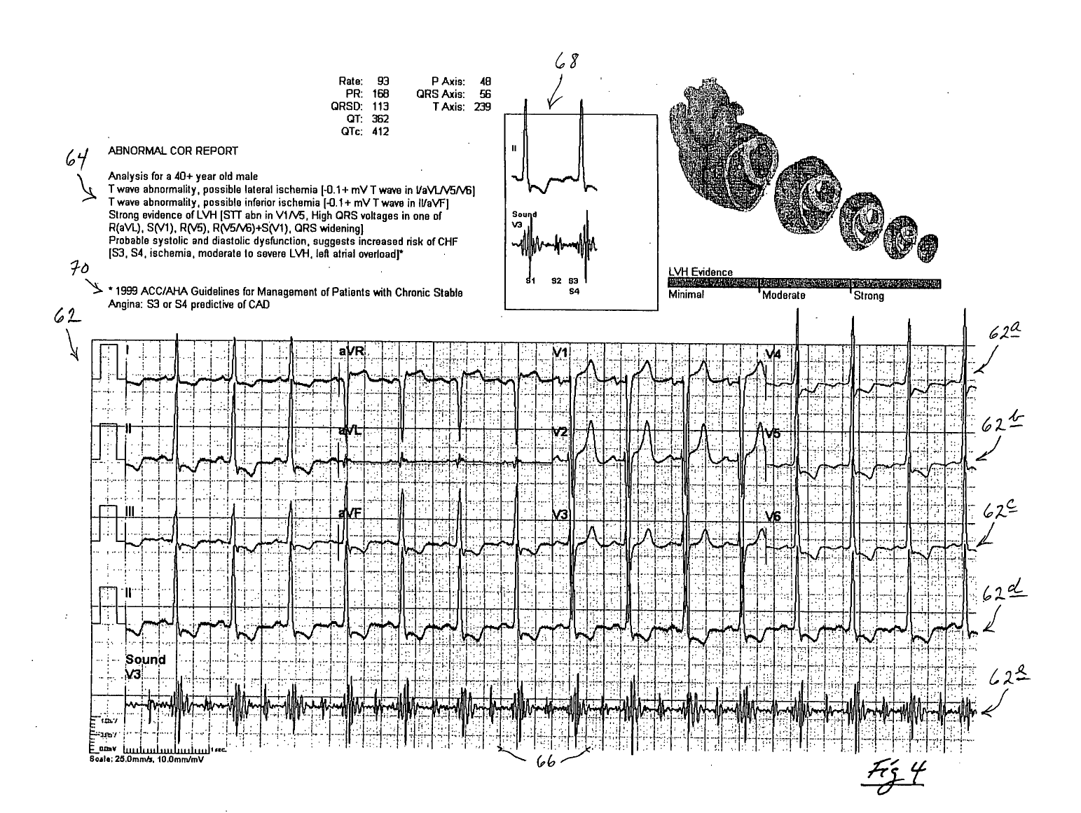

Deflecting attention for just a moment now to FIGS. 2 and 3, FIG. 2 provides one way of looking at performance of the invention via a pair of blocks 48, 50, labeled, respectively, COLLECT ECG/AUDIO DATA, and COMPARE WITH AUTHORITATIVE GUIDELINES AND REPORT. FIG. 2 thus focuses attention on one high-level view regarding the practice of the present invention. This view highlights the ability of the present invention to spring from collected ECG and audio data to an output report which becomes linked with authoritative, confirmatory interpretation guidelines.

FIG. 3, in five blocks 52, 54, 56, 58, 60, presents another high-level view of the invention. Block 52 is labeled COLLECT ECG/AUDIO DATA and represents the activities of sensor/transducer structures, such as those represented in FIG. 1 at 14, 16. Block 54 represents the operations, effectively, of blocks 18, 20 in FIG. 1, with block 54 being labeled PROCESS AND PRESENT IN VIEWABLE FORM. Blocks 56, 58, labeled ISOLATE AND PRESENT SNIPPET, and RELATE FINDINGS TO GUIDELINES AND PRESENT REPORT, respectively, reflect, in a high-level fashion, the operations of blocks 22-30, inclusive, in FIG. 1. Block 60 represents the fact that, in accordance with practice of the present invention, the isolated ECG and audio snippet which becomes display-presented is, effectively, representative of a single heartbeat of a subject's heart.

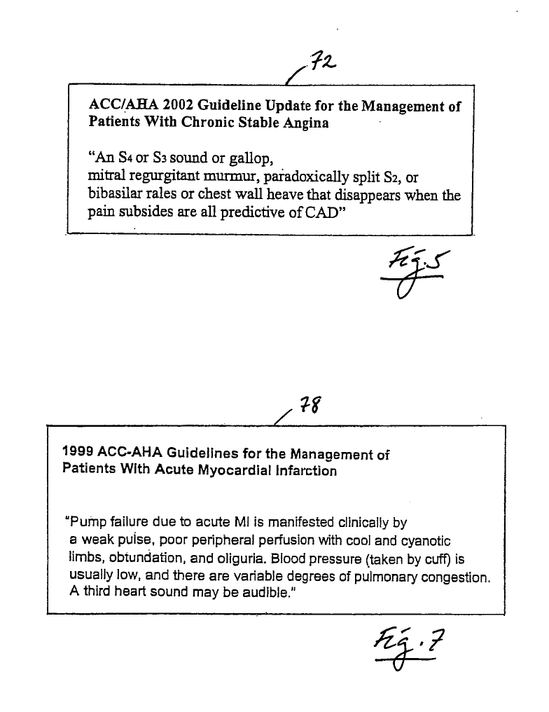

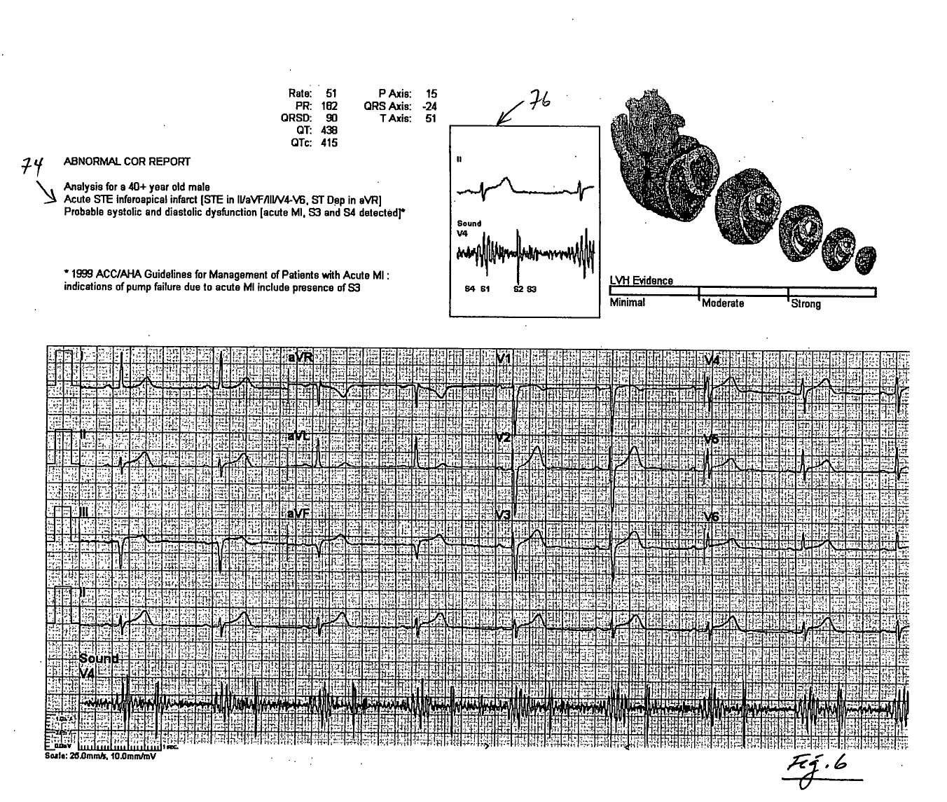

FIG. 4-7, inclusive, to which we now address attention, illustrate two different strip-chart-type reports that have been generated by practice of the invention as illustrated and described in and with respect to FIGS. 1-3, inclusive. As was mentioned above in the description of the drawing figures, FIGS. 4 and 5 are linked to one another, as is also true with respect to FIGS. 6 and 7.

Beginning with FIGS. 4 and 5, indicated generally at 62 in FIG. 4 is a graphical analog waveform display including five basically horizontal rows of analog waveform information shown at 62a, 62b, 62c, 62d and 62e. Those familiar with conventional strip-chart ECG waveform presentations will recognize that the analog traces shown at 62a, 62b, 62c collectively present short-time representations of the ECG waveforms collected, respectively, from each of the conventional twelve ECG leads. The waveform shown at 62d has been selected to present only the ECG information derived from conventional ECG lead number II (two). The waveform shown at 62e is the sound waveform which has been collected from conventionally recognized site V3 in the anatomy. It should be understood, however, that such a sound waveform may come from a site other than site V3, with the particular site choice for this information being based primarily on sound-signal clarity and ease of differentiation from noise.

These several waveforms collectively represent information which has been collected as illustrated in FIG. 1 from subject anatomy 12. It is from this waveform information that processing is performed to develop an interpretation, and indicated generally in text form at 64 in FIG. 4 is the displayed output of a textual description of an interpretation which has been performed on the data shown in analog waveform style in FIG. 4.

Looking toward the bottom of FIG. 4, one observes the presence of two markers 66 which effectively laterally bracket just slightly more than one full heartbeat of the associated human subject. These markers have been placed in the location shown to identify the small time portion of what is pictured in FIG. 4 which has been selected to furnish a good graphical illustration of the sound and ECG waveforms that best “picture” the interpretation produced by block 18 (FIG. 1), and the interpretation text shown at 64. Further, it is from this region of what is shown in FIG. 4 that the ECG waveform from lead II, and the relevant time-associated portion of the presented time waveform, have been selected to present what has been referred to herein as a graphical, combined ECG and sound snippet 68 which is shown centered near the top of FIG. 4. One will notice in this snippet that the locations of heart sounds S1, S2, S3, S4 have been marked, and thus indicated in the displayed snippet to be present.

If one takes a careful look at textual interpretation 64 in FIG. 4, one will notice an asterisk appearing at the bracket which closes out that report. This asterisk makes reference to an authoritative guideline which has been found in guideline database 26 through comparison activity performed in Block 28 in FIG. 1. This guideline, in abbreviated form, is shown at 70 in FIG. 4, and in full form in block 72 in FIG. 5. While there was not adequate room in the drawings herein to incorporate block 72 on to the plate of drawings containing FIG. 4, in the actual output of a strip-chart display report like that shown in FIG. 4, the same would preferably be printed in such as fashion that the content of block 72 would appear along with the graphical information seen in FIG. 4. Block 72 herein thus presents the text of a specific confirmatory guideline which relates to the interpretation that has been performed, and described in FIG. 4, and which is illustrated as a graphical snippet at 68 in FIG. 4.

FIGS. 6 and 7 are related to one another in the same manner that FIGS. 4 and 5 are related to each other. In FIG. 6, a textual interpretation is shown at 74, and a related graphical snippet is shown at 76. In a block 78 in FIG. 7, there is presented the full text of an authoritative, specific guideline drawn from database 26, which guideline was found during a comparison of the associated performed ECG and sound interpretation which is expressed at 74 and 76 in FIG. 6.

Thus, a unique methodology involving a unique display output presentation of related ECG and sound information is proposed and offered by practice of the present invention. Output display, such as that illustrated in FIGS. 4-7, inclusive, placed in the hands of an appropriately skilled clinician, offers a strikingly comprehensive and informative ECG and heart-sound-associated interpretation of the condition of a subject person's heart. By combining the illustrated representations of collected sound and ECG waveforms, by isolating an interpretation-based snippet, such as those shown at 68, 76 in FIGS. 4 and 6, respectively, and by marking the located heart sounds (the S sounds) in these graphical snippets, a clinician is given a powerful visual tool for assessing heart condition.

As has been suggested earlier herein, there are several high-level ways in which one can view the unique methodology of the present invention—a methodology which features a computer-based approach for presenting correlated ECG and heart-associated sound information relating to a selected subject's heart.

In one manner of visualizing the invention, this methodology includes the steps of (a) collecting time-base-related, associated, selected-subject ECG and sound information, (b) processing this collected information and generating from it a reportable interpretation, and (c) following such processing, producing an integrated report which commonly shows, in a focused time-based snippet, time-related ECG and sound regions drawn from the collected data which regions have played a role in the generating of the mentioned reportable interpretation.

From another perspective, the steps of the invention can be seen to include (a) collecting time-base-related, associated, selected-subject ECG and sound information, (b) processing such collected information and generating from it a reportable interpretation, (c) comparing the generated interpretation with a recognized, authoritative body of ECG and heart-sound clinical interpretation guidelines thus to identify specific guideline text which is relevant to the generated interpretation, and (d) producing an integrated report which shows at least both the generated interpretation and the identified guideline text.

Still a further high-level way of viewing the steps of the invention is to observe that it includes the steps of (a) collecting subject-specific, time-related ECG and heart-associated sound information, (b) effectively comparing that collected information with a recognized, authoritative body of ECG and heart-sound clinical interpretation guidelines, and (d) producing a report which describes the results of such comparing activity.

Yet another high-level way of viewing the steps of the invention is to describe them as including (a) collecting subject-specific, time-related ECG and heart-associated sound information, and (b), thereafter presenting, in a common-display manner, at least portions of this ECG and sound information along with (1) a stated interpretation derived from the information, coupled with (2).the presentation of a related, authoritative clinical-interpretation guideline.

Thus, while various facets of a preferred manner of practicing the invention have been illustrated and described herein, we appreciate that variations and modifications may be made without departing from the spirit of the invention.

We claim:

1. A computer-based method for presenting correlated ECG and heart-associated sound information regarding a selected subject's heart comprising
collecting time-base-related, associated, selected-subject ECG and sound information,
processing such collected information and generating therefrom a reportable interpretation, and
following said processing, producing an integrated report which commonly shows, in a focused, time-based snippet, time-related regions from each of the collected ECG information and the collected sound information, which regions played a role in said generating of the mentioned reportable interpretation.

2. The method of claim 1, wherein said producing includes creating a printed chart-like display.

3. The method of claim 1, wherein said producing includes creating a screen-viewable display.

4. The method of claim 1, wherein said producing includes preparing a display-relevant database.

5. The method of claim 1, wherein the mentioned focused snippet essentially illustrates a single beat of the selected subject's heart.

6. The method of claim 1, wherein said collecting, processing, generating, and producing are performed on a real-time basis.

7. The method of claim 1 which further comprises, following said generating, comparing the generated interpretation with a recognized, authoritative body of ECG and heart-sound clinical interpretation guidelines to identify guideline text which is relevant to the generated interpretation, and said producing includes presenting in the integrated report both the generated interpretation and the associated, relevant guideline text.

8. A computer-based method for presenting correlated ECG and heart-associated sound information regarding a selected subject's heart comprising
collecting time-base-related, associated, selected-subject ECG and sound information,
processing such collected information and generating therefrom a reportable interpretation,
comparing the generated interpretation with a recognized, authoritative body of ECG and heart-sound clinical interpretation guidelines to identify guideline text which is relevant to the generated interpretation, and
producing an integrated report which commonly shows at least (a) the generated interpretation, and (b) the identified guideline text.

9. The method of claim 8, wherein said producing results in such a report which additionally shows graphically (a) the collected ECG information, (b) the collected sound information, and (c) a focused, time-based snippet of related regions from each of the collected ECG information and the collected sound information which regions played a role in said generating of the mentioned reportable interpretation.

10. The method of claim 9, wherein said producing includes creating a printed chart-like display.

11. The method of claim 9, wherein said producing includes creating a screen-viewable display.

12. The method of claim 9, wherein said producing includes preparing a display-relevant display database.

13. The method of claim 9, wherein the mentioned focused snippet essentially illustrates a single beat of the selected subject's heart.

14. The method of claim 8, wherein said collecting, processing, generating, comparing, and producing are performed on a real-time basis.

15. A computer-based method for presenting correlated ECG and heart-associated sound information regarding a selected subject's heart comprising
collecting subject-specific, time-related ECG and heart-associated sound information,
effectively comparing that collected information with a recognized, authoritative body of ECG and heart-sound clinical interpretation guidelines, and
producing a report which describes the results of said comparing.

16. A computer-based method for presenting correlated ECG and heart-associated sound information regarding a selected subject's heart comprising
collecting subject-specific, time-related ECG and heart-associated sound information, and
thereafter presenting, in a common-display manner, at least portions of this ECG and sound information along with (a) a stated heart-condition interpretation derived from the information, coupled with (b) the presentation of a related, authoritative clinical-interpretation guideline.
